(19) United States
(12) Patent Application Publication
(10) Pub. No.: US 2021/0353196 A1
(43) Pub. Date: Nov. 18, 2021

(54) METHOD AND DEVICE FOR EVALUATING PHYSICAL STAMINA INDICATORS IN RUNNING EXERCISE

(71) Applicant: Guangdong Coros Sports Technology Co., Ltd., Dongguan (CN)

(72) Inventors: Xin Liu, Dongguan (CN); Xuan Rao, Dongguan (CN); Yu Tang, Dongguan (CN); Muyi Huang, Dongguan (CN); Haotian Liu, Dongguan (CN)

(21) Appl. No.: 17/109,104

(22) Filed: Dec. 1, 2020

(30) Foreign Application Priority Data
May 13, 2020 (CN) 202010405185.X

(51) Int. Cl. A61B 5/22; A61B 5/00; A63B 24/00; A61B 5/0205; G16H 50/30; G16H 20/30; G16H 70/20; G16H 50/70; G16H 10/60; G16H 40/67; G06F 16/2457

(52) U.S. Cl. CPC A61B 5/224; A61B 5/02405; A63B 24/0062; A61B 5/4866; A61B 5/7278; A61B 5/0205; G16H 50/30; G16H 20/30; G16H 70/20; G16H 50/70; G16H 10/60; G16H 40/67; G06F 16/2457; A63B 2220/30; A63B 2024/0068; A61B 5/4884

(57) ABSTRACT

A method for evaluating physical stamina indicators in running exercise includes collecting different exercise condition data, establishing a second and a third model equations, calculating a first and a second standard paces, calculating a first and a second physical stamina levels according to the paces, and finally confirming the physical stamina category. The accuracy of the evaluation results is high for the reference.

### RELATED APPLICATIONS

This application claims the benefit of priority to Chinese invention applications No. 202010405185.X filed on May 13, 2020, which is hereby incorporated by reference in its entirety.

### FIELD OF THE INVENTION

The present invention relates to technical field of physical stamina evaluation, and in particular to a method and equipment for evaluating physical stamina indicators including physical stamina categories and physical stamina conditions in running exercise.

### BACKGROUND OF THE INVENTION

Running becomes more and more popular as running is not restricted by venues, equipment and other factors. For runners, it's important to set a reasonable running pace according to their own physical stamina. If the pace is too fast, a full course may be not completed due to physical exhaustion; if the pace is too slow, effect training and a desired competition result may be not obtained. In addition, for some runners who are good at the half-marathon distance, it's difficult to configure their own physical strengths to complete a running pace for 5-kilometer distance or full marathon distance, due to the unknown physical stamina condition. Moreover, the evaluation of their own physical stamina and endurances has great reference significance for formulating training targets for runners. In the prior art, the assessment of physical stamina is based on the experience of the evaluator for qualitative analysis, which is difficult for some amateur runners. Therefore, it is necessary to improve the existing physical stamina indicator evaluation method so that any runner can easily know his own physical stamina condition.

### SUMMARY OF THE INVENTION

One of the objectives of the present invention is to provide a method for evaluating physical stamina indicators in running exercise to solve the above technical problems, so as to make it convenient for users to know their physical stamina category.

To achieve the above objectives, the present invention provides method for evaluating physical stamina indicators in running exercise, comprising a method for evaluating a physical stamina category which comprises:

defining a closed numerical interval D1, and using values in the numerical interval D1 to quantify a physical stamina level;

establishing a first model equation corresponding to a certain standard schedule, where the first model equation reflects a relationship between a physical stamina level and a pace under the standard schedule;

acquiring a first standard pace that is relevant to the standard schedule and reflects user's running level in recent N days, and obtaining a first physical stamina level that reflects user's exercise performance through the first model equation;

defining several different physical stamina categories, and establishing a physical stamina category query table which records the physical stamina categories corresponding to users with different physiological characteristics and different physical stamina categories; and

querying the physical stamina category query table according to user's physiological characteristics and the first physical stamina level that is calculated, thereby obtaining user's physical stamina category.

In comparison with the prior art, when the above evaluation method is used to evaluate the physical stamina category, the user's quantified first physical stamina level will be calculated first, and then the corresponding physical stamina category can be confirmed according to the first physical stamina level. In such a way, the evaluation result is highly accurate and can be referenced, and for the user, the intuitive physical stamina category can be obtained easily by only providing the relevant basic data such as the first standard pace etc., the operation is convenient.

Preferably, the first standard pace is acquired by following steps:

S30, collecting a real-time heart rate and a real-time exercise speed of the user during an exercise in recent N days, and establishing a second model equation that reflects a relationship between user's heart rate and pace;

S31, obtaining a reserve heart rate ratio of the user with suitable exercise intensity under the standard schedule;

S32, obtaining user's maximum heart rate and resting heart rate, and obtaining user's exercise heart rate for the standard schedule according to a calculation formula of the reserve heart rate ratio; and

S33: substituting the exercise heart rate into the second model equation to obtain the user's first standard pace for the standard schedule.

Preferably, the real-time heart rate and the real-time exercise speed are collected by following steps:

S300, determining whether the user's single continuous exercise duration T1 is greater than a preset duration T2, if yes, performing step S301; otherwise, return;

S301, in a certain movement period, according to time sequence, extracting the real-time heart rate and the real-time exercise speed in each continuous unit time period and determining whether the real-time heart rate and the real-time exercise speed meet following conditions:

a first condition: fluctuation of the real-time heart rate is within a preset value B1:

a second condition: fluctuation of the real-time exercise speed is within a preset value V1;

a third condition: the real-time reserve heart rate ratio is within a preset interval D2;

If yes, applying the real-time heart rate and real-time exercise speed in the unit time period as collected data.

Preferably, the step S31 comprises:

obtaining user's physiological characteristics data and maximal oxygen consumption data;

preparing a reserve heart rate ratio query table corresponding to the standard schedule, the reserve heart rate ratio query table adapted for recording reserve heart rate ratios corresponding to users who have different physiological characteristics and maximal oxygen consumption for the standard schedule; and

querying the reserve heart rate ratio query table according to the user's physiological characteristic data and the maximal oxygen consumption data, thereby obtaining user's reserve heart rate ratio for the standard schedule.

The maximal oxygen consumption is obtained by following steps:

obtaining the user's age, gender, and weight;

collecting the real-time heart rate and real-time exercise speed of the user during exercise;

selecting a characteristic time period with a preset time length from the user's current exercise duration, and calculating a characteristic average heart rate and a characteristic average speed in the characteristic time period using the real-time heart rate and the real-time exercise speed as basic data;

calculating the current user's maximal oxygen consumption Vₒ_{₂}max according to a following formula:

$${V}_{{o}_{2} max} = A + P 1 * S - P 2 * G + P 3 * V * \frac{P 4}{B} - C * \frac{{HR}_{chara} - {HR}_{rest}}{{HR}_{max} - {HR}_{rest}} - \frac{2 ( a - 26 )}{5} ,$$

wherein A is a constant from 40 to 50, P1 is a constant from 7 to 8, S is a gender constant, male is equal to 1, and female is equal to 0; P2 is a constant from 0.1 to 0.2, G is the user's weight; P3 is 4 to 5, V is the characteristic average speed. P4 is a constant from 3 to 4, B is a constant from 1 to 2; C is a constant from 15 to 20, HR_{chara} is the characteristic average heart rate, HRᵣₑₛₜ is the resting heart rate in a quiet state for the user, HRₘₐₓ is the maximum heart rate; and a is the user's age.

Preferably, the first model equation is established by following steps:

collecting different running paces of different athletes with different levels corresponding to the standard schedule, according to a positive correlation between the physical stamina categories and the running paces, matching the running paces with the physical stamina categories to form different data pairs, processing the data pairs and establishing a regression model between physical stamina and pace to obtain the first model equation corresponding to the standard schedule.

Preferably, the method further includes a method for evaluating physical stamina condition comprising:

defining several different evaluation characteristics that indicate physical stamina conditions;

acquiring user's a second standard pace that is relevant to the standard schedule and reflects user's running level in recent M days, and obtaining a second physical stamina level that reflects user's athletic ability through the first model equation, wherein M>N;

calculate a physical stamina difference DV between the second physical stamina level and the first physical stamina level;

establishing a physical stamina condition evaluation table which records the evaluation characteristics corresponding to users with different physical stamina differences DV; and

querying the physical stamina condition evaluation table according to user's physical stamina difference DV, thereby obtaining user's evaluation characteristics representing the physical stamina condition.

Preferably, M is equal to 28, and N is equal to 7.

Further, the present invention provides a device for evaluating a physical stamina indicator in running exercise, comprising a physical stamina category evaluation unit which comprises:

a physical stamina level quantification module, configured to quantify a physical stamina level by using a closed numerical interval D1 that is self-defined;

a first model generation module, configured to establish a first model equation corresponding to a certain standard schedule, where the first model equation reflects a relationship between a physical stamina level and a pace under the standard schedule:

a first calculation module, configured to acquire user's a first standard pace that is relevant to the standard schedule and reflects user's running level in recent N days, and obtain a first physical stamina level that reflects user's exercise performance through the first model equation; and

a physical stamina category query module, configured to query a physical stamina category query table according to the first physical stamina level, thereby obtaining user's physical stamina category.

Preferably, the physical stamina category evaluation unit further comprises:

a second model generation module, configured to establish a second model equation reflecting a relationship between user's heart rate and pace, according to a real-time heart rate and a real-time exercise speed of the user during the exercise in recent N days;

a second calculation module, configured to calculate user's exercise heart rate for the standard schedule according to user's maximum heart rate, resting heart rate and reserve heart rate ratio; and

a third calculation module, configured to calculate user's first standard pace for the standard schedule according to the exercise heart rate and the second model equation.

Preferably, during the establishment of the second model equation, the real-time heart rate and the real-time exercise speed are collected by following steps:

S300, determining whether the user's single continuous exercise duration T1 is greater than a preset duration T2, if yes, performing step S301; otherwise, return:

S301, in a certain movement period, according to time sequence, extracting the real-time heart rate and the real-time exercise speed in each continuous unit time period and determining whether the real-time heart rate and the real-time exercise speed meet following conditions:

a first condition: fluctuation of the real-time heart rate is within a preset value B;

a second condition: fluctuation of the real-time exercise speed is within a preset value V1;

a third condition: the real-time reserve heart rate ratio is within a preset interval D2:

If yes, applying the real-time heart rate and real-time exercise speed in the unit time period as collected data.

Preferably, the step S31 comprises:

obtaining user's physiological characteristics data and maximal oxygen consumption data;

preparing a reserve heart rate ratio query table corresponding to the standard schedule, the reserve heart rate ratio query table adapted for recording reserve heart rate ratios corresponding to users who have different physiological characteristics and maximal oxygen consumption for the standard schedule; and

querying the reserve heart rate ratio query table according to the user's physiological characteristic data and the maximal oxygen consumption data, thereby obtaining user's reserve heart rate ratio for the standard schedule.

The maximal oxygen consumption is obtained by following steps:

obtaining the user's age, gender, and weight;

collecting the real-time heart rate and real-time exercise speed of the user during exercise:

selecting a characteristic time period with a preset time length from the user's current exercise duration, and calculating a characteristic average heart rate and a characteristic average speed in the characteristic time period using the real-time heart rate and the real-time exercise speed as basic data:

calculating the current user's maximal oxygen consumption Vₒ_{₂}max according to a following formula:

$${V}_{{o}_{2} max} = A + P 1 * S - P 2 * G + P 3 * V * \frac{P 4}{B} - C * \frac{{HR}_{chara} - {HR}_{rest}}{{HR}_{max} - {HR}_{rest}} - \frac{2 ( a - 26 )}{5} ,$$

wherein A is a constant from 40 to 50, P1 is a constant from 7 to 8, S is a gender constant, male is equal to 1, and female is equal to 0; P2 is a constant from 0.1 to 0.2, G is the user's weight; P3 is 4 to 5, V is the characteristic average speed, P4 is a constant from 3 to 4, B is a constant from 1 to 2; C is a constant from 15 to 20, HR_{chara} is the characteristic average heart rate, HRᵣₑₛₜ is the resting heart rate in a quiet state for the user, HRₘₐₓ, is the maximum heart rate; and a is the user's age.

Preferably, the first model equation is established by following steps:

collecting different running paces of different athletes with different levels corresponding to the standard schedule, according to a positive correlation between the physical stamina categories and the running paces, matching the running paces with the physical stamina categories to form different data pairs, processing the data pairs and establishing a regression model between physical stamina and pace to obtain the first model equation corresponding to the standard schedule.

Preferably, the device further comprises a physical stamina condition evaluation unit which includes a fourth calculation module, a fifth calculation module, and a physical stamina condition query module. Specifically, the fourth calculation module is configured to acquire user's a second standard pace that is relevant to the standard schedule and reflects user's running level in recent M days, and calculate a second physical stamina level that reflects user's athletic ability through the first model equation, wherein M>N. The fifth calculation module is configured to obtain a physical stamina difference DV between the second physical stamina level and the first physical stamina level. The physical stamina condition query module is configured to obtain the current user's evaluation characteristics indicating the physical stamina condition by querying the physical stamina condition evaluation table according to the current physical stamina difference DV.

The present invention also provides an apparatus for evaluating physical stamina indicators in running exercise, including one or more processors; one or more memories; and one or more programs, stored in said one or more memories and configured to be executed by said one or more processors, for executing the method for evaluating physical stamina indicators in running exercise mentioned above.

The present invention also provides a readable storage medium in which programs are stored. When the programs are executed by the processor, to implement the method for evaluating physical stamina indicators in running exercise as described above.

### BRIEF DESCRIPTION OF THE DRAWINGS

The accompanying drawings facilitate an understanding of the various embodiments of this invention. In such drawings:

FIG. 1 is a schematic flow chart of a method for evaluating physical stamina categories according to an embodiment of the present invention;

FIG. 2 is a schematic flowchart of a method for obtaining a first standard pace in an embodiment of the present invention;

FIG. 3 is a schematic flow chart of a method for evaluating physical stamina categories and physical stamina conditions according to an embodiment of the invention; and

FIG. 4 is a schematic diagram of the principle structure of an evaluation device in an embodiment of the present invention.

### DETAILED DESCRIPTION OF ILLUSTRATED EMBODIMENTS

A distinct and full description of the technical solution of the present invention will follow by combining with the accompanying drawings.

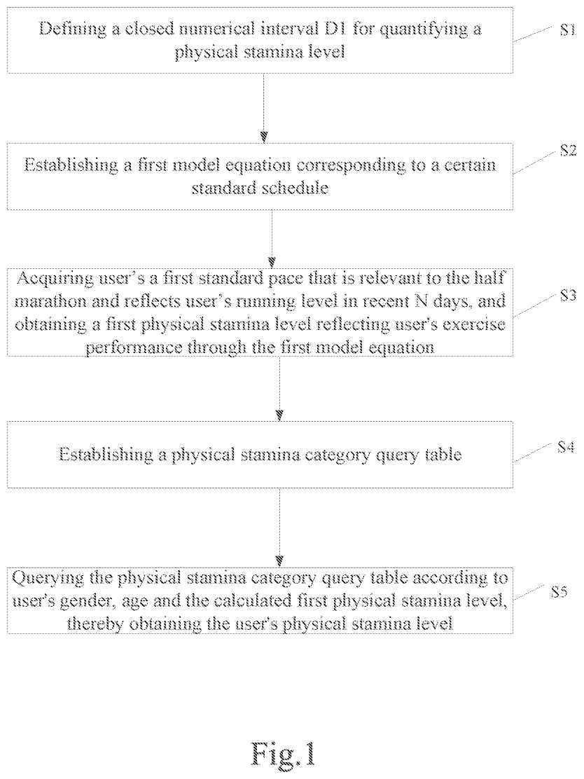

Referring to FIG. 1, the present invention provides a method for evaluating physical stamina indicators in running exercise including a method for evaluating a physical stamina category and a method for evaluating a physical stamina condition.

As shown in FIG. 1, the method for evaluating a physical stamina category includes the following steps (there is no order limit).

S1, defining a closed numerical interval D1, and using values in the numerical interval D1 to quantify a physical stamina level. Since there is currently no unified quantification standard for physical stamina levels, in order to facilitate statistical calculation of this reference variable, a self-defined numerical interval D1 is used in this embodiment. Specifically, the numerical interval D1 is [0,100], that is, the minimum physical stamina level is defined as 0, and the maximum physical stamina level is defined as 100.

S2, establishing a first model equation corresponding to a certain standard schedule, where the first model equation reflects a relationship between a physical stamina level and a pace under the standard schedule. In running exercise, the half marathon has the largest number of participants, so half marathon is used as the standard schedule in this embodiment. The method for establishing the first model equation can be obtained by checking theoretical data, or by fitting the statistical data of athletes of different running levels in the half marathon with previous marathons (the fitting process will be detailed in the following embodiments).

S3, acquiring user's a first standard pace that is relevant to the half marathon and reflects user's running level in recent N days, and obtaining a first physical stamina level reflecting user's exercise performance through the first model equation. The first standard pace can be directly provided by the user, or obtained by detecting the user's daily exercise data indicator.

S4, defining several different physical stamina categories, and establishing a physical stamina category query table, as shown in Table 1 and Table 2. The physical stamina category query table is for recording the physical stamina categories corresponding to users with different physiological characteristics and different physical stamina levels. For example, the physical characteristics include gender and age, etc. In this embodiment, the physical stamina category is classified into four categories, including novice, advanced, excellent, and elite, which is not limited. For example, the physical stamina category can also be classified into four categories A, B, C, and D.

TABLE 1

| | | | |
|---|---|---|---|
| Novice | Advanced | Excellent | Elite |

| | |
|---|---|
| Age (Male) | Physical stamina level |

| | | | | |
|---|---|---|---|---|
| 29 and below | 0-40 | 41-60 | 61-70 | 71-100 |
| 30-39 | 0-39 | 40-59 | 60-69 | 70-100 |
| 40-49 | 0-38 | 39-58 | 59-68 | 69-100 |
| 50-59 | 0-36 | 37-56 | 57-66 | 67-100 |
| 60 and above | 0-33 | 34-53 | 54-63 | 64-100 |

TABLE 2

| | | | |
|---|---|---|---|
| Novice | Advanced | Excellent | Elite |

| | |
|---|---|
| Age (Female) | Physical stamina level |

| | | | | |
|---|---|---|---|---|
| 29 and below | 0-32 | 33-52 | 53-62 | 63-100 |
| 30-39 | 0-31 | 32-51 | 52-61 | 62-100 |
| 40-49 | 0-30 | 31-50 | 51-60 | 61-100 |
| 50-59 | 0-28 | 29-48 | 49-58 | 59-100 |
| 60 and above | 0-25 | 26-45 | 46-55 | 56-100 |

S5, querying the physical stamina category query table according to user's gender, age and the calculated first physical stamina level, thereby obtaining the user's physical stamina level. Table 1 is applied if the user is male while Table 2 is applied if the user is female.

The first model equation is established as follows:

collecting different running paces of athletes with different levels in the half-marathon race, according to a positive correlation between the physical stamina level and the running pace, matching the different running paces with the different physical stamina levels to form data pairs. For example, matching the highest pace of 2:45 (currently the highest record for a half marathon) with the highest physical stamina level of 100. The matching data pairs are shown in Table 3:

TABLE 3

| Physical stamina level | Pace (Minute/km) |
|---|---|
| 100 | 2:45 |
| 90 | 3:23 |
| 80 | 4:06 |
| 70 | 4:46 |
| 60 | 5:29 |
| 50 | 6:20 |
| 40 | 6:54 |
| 30 | 7:30 |
| 20 | 8:05 |
| 10 | 8:55 |
| 0 | 9:30 |

Then the different data pairs in Table 3 are processed, that is, each physical stamina level and the corresponding half-marathon pace are drawn in a rectangular coordinate system. Through the method of data fitting, the first model equation that matches the half marathon is obtained: y₁=k₁*x₁+b₁, wherein k₁ and b₁ are constants, y₁ represents the physical stamina level the user can show, and x₁ represents the half-marathon pace that the user can show.

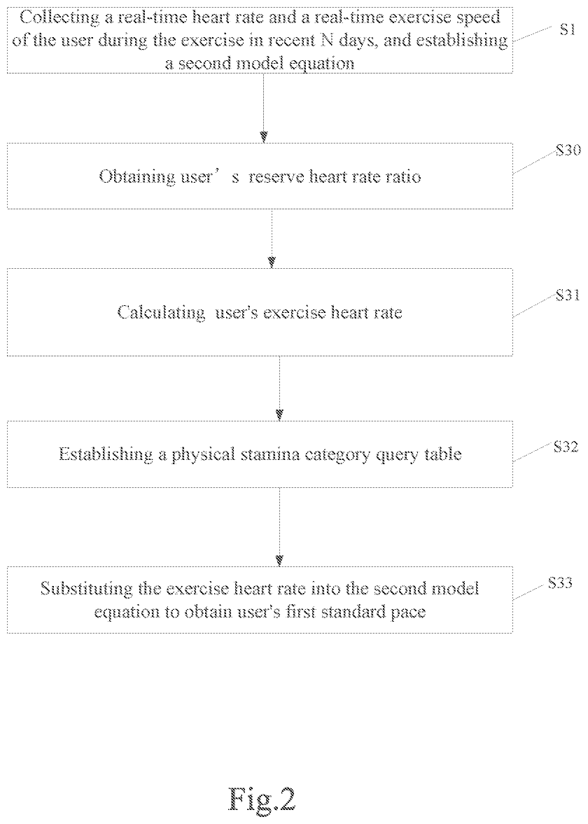

Furthermore, as shown in FIG. 2, the first standard pace is acquired by following steps:

S30, collecting a real-time heart rate and a real-time exercise speed of the user during the exercise in recent N days, and establishing a second model equation reflecting the relationship between the user's heart rate and the pace;

S31, obtaining a reserve heart rate ratio of the user with suitable exercise intensity under the standard schedule;

S32, obtaining the user's maximum heart rate and resting heart rate, and obtaining user's exercise heart rate for the standard schedule according to a calculation formula of the reserve heart rate ratio; and

S33: substituting the exercise heart rate into the second model equation to obtain user's first standard pace for the standard schedule.

In the above embodiment, the reserve heart rate ratio ρ reflects the user's exercise intensity, and its calculation formula is:

$$ρ = \frac{{HR}_{exercise} - {HR}_{rest}}{{HR}_{max} - {HR}_{rest}} ,$$

wherein HR_{exercise} represents user's exercise heart rate, and HRₘₐₓ represents user' maximum heart rate, HRᵣₑₛₜ represents use's resting heart rate in a awake and quiet state. Specifically, the maximum heart rate and resting heart rate can be detected by a portable detection device, or manually set by the users if he/she knows their own maximum heart rate and resting heart rate. In addition, the maximum heart rate can also be calculated by the user's age, ie HRₘₐₓ=208−0.7*a, where a is the use's age. Similarly, the reserve heart rate ratio ρ can also be manually set if the user knows his suitable exercise intensity for the half marathon.

When collecting exercise condition data, in order to filter some data irrelevant to the exercise to the greatest extent to improve the prediction accuracy, the collecting method of the exercise condition data includes:

S300, determining whether the user's single continuous exercise duration T1 is greater than a preset duration T2, if yes, performing step S301; otherwise, return;

S301, in a certain movement period, according to time sequence, extracting the real-time heart rate and the real-time exercise speed in each continuous unit time period and determining whether the real-time heart rate and the real-time exercise speed meet following conditions:

a first condition: fluctuation of the real-time heart rate is within a preset value B1;

a second condition: fluctuation of the real-time movement speed is within a set value V1;

a third condition: the real-time reserve heart rate ratio is within a preset interval D2;

If yes, applying the real-time heart rate and real-time exercise speed in the unit time period as collected data; otherwise, the data is discarded.

Specifically, T2 is 10 minutes, B1 is 10 bpm, V1 is 0.5 m/s, and D2 is [50%, 90%], then when collecting exercise condition data through the electronic device, the method includes firstly determining whether the user's continuous exercise duration is greater than 10 minutes, if no, no data in the exercise time period is collected; if yes, extracting the real-time heart rate and the real-time exercise speed in each continuous unit time period for analysis, in a certain movement period, according to time sequence. The unit time period here is 2 minutes as an example. The real-time heart rate and the real-time exercise speed are extracted as follows: respectively extracting data iⁿ the 1^{st} second to ^{th}e 120^{th} second the 1^{st} second to ^{th}e 121^{st} second the 1^{st} second to ^{th}e 122^{nd} second, . . . for example. For exercise condition data within any 2 minutes, data will be collected only if the fluctuation of real-time heart rate does not exceed 10 bpm, the fluctuation of real-time exercise speed does not exceed 0.5 m/s, and the real-time reserve heart rate ratio calculated based on the real-time heart rate value is between 50% to 90, otherwise, the data will be discarded.

After the required exercise condition data is collected, an arithmetic average algorithm is used to average the real-time heart rate and real-time exercise speed in each 2-minute period to obtain a unit average heart rate and a unit average exercise speed. And then several groups of unit average heart rates and unit average speeds are depicted in a rectangular coordinate system to fit a second model equation through the fitting method: y₂=k₂*x₂+b₂, wherein k₂ and b₂ are constants, and y₂ indicates the exercise heart rate that the user can show, and x₁ indicates the running pace that the user can show. When the user's exercise heart rate is obtained through the reserve heart rate ratio, the exercise heart rate is substituted into the second model equation y₂ to obtain the first standard pace for the half marathon reflecting the user's running level in the recent 7 days, and then user's first physical stamina level can be obtained through the second model equation.

Further, the step S31 of obtaining a reserve heart rate ratio of the user with suitable exercise intensity under the half marathon includes:

obtaining the user's physiological characteristics data such as age and gender, and the maximal oxygen consumption data which may be generated according to the physiological characteristics data or set manually:

preparing the reserve heart rate ratio query table corresponding to the standard schedule. For example, the tables 4 and 5 shows different reserve heart rate ratios corresponding to users who have different physiological characteristics and different maximal oxygen consumptions (VO₂max) for the standard schedule; and

querying the reserve heart rate ratio query table according to the user's physiological characteristic data and the maximal oxygen consumption data, thereby obtaining the user's reserve heart rate ratio for the standard schedule.

TABLE 4

| | | 78% (any | |
|---|---|---|---|
| | | percentage | |
| | | between | |
| | 75% | 75% and 80%) | 80% |
| Age | VO₂ max | VO₂ max | VO₂ max |
| (Male) | (Area I) | (Area II) | (Area III) |
| 29 and below | 45 and below | 46-59 | 60 and above |
| 30-34 | 43 and below | 44-57 | 58 and above |
| 35-39 | 41 and below | 42-55 | 56 and above |
| 40-44 | 39 and below | 40-53 | 54 and above |
| 45-49 | 37 and below | 38-51 | 52 and above |
| 50-54 | 35 and below | 36-49 | 50 and above |
| 55-59 | 33 and below | 34-47 | 48 and above |
| 60-64 | 31 and below | 32-45 | 46 and above |
| 65 and above | 29 and below | 30-43 | 43 and above |

TABLE 5

| | | 78% (any | |
|---|---|---|---|
| | | percentage | |
| | | between | |
| | 75% | 75% and 80%) | 80% |
| Age | VO₂ max | VO₂ max | VO₂ max |
| (Female) | (Area I) | (Area II) | (Area III) |
| 29 and below | 38 and below | 39-52 | 53 and above |
| 30-34 | 36 and below | 37-50 | 51 and above |
| 35-39 | 34 and below | 35-48 | 49 and above |
| 40-44 | 32 and below | 33-46 | 47 and above |
| 45-49 | 30 and below | 31-44 | 45 and above |
| 50-54 | 28 and below | 29-42 | 43 and above |
| 55-59 | 26 and below | 27-40 | 41 and above |
| 60-64 | 24 and below | 25-38 | 39 and above |
| 65 and above | 22 and below | 23-36 | 37 and above |

Therefore, if the user is a male, user's reserve heart rate ratio for the half marathon can be obtained by querying Table 4 according to the user's age and VO₂max data. If the user is a female, user's reserve heart rate ratio for the half marathon can be obtained by querying Table 5 according to the user's age and VO₂max data.

Preferably, the maximal oxygen consumption is obtained by following steps:

obtaining the user's age, gender, weight, resting heart rate and maximum heart rate;

collecting the real-time heart rate and real-time exercise speed of the user during exercise;

selecting a characteristic time period with a preset time length from the user's current exercise duration, and calculating a characteristic average heart rate and a characteristic average speed in the characteristic time period using the real-time heart rate and the real-time exercise speed as basic data; and

calculating the current use's maximal oxygen consumption VO₂max according to a following formula:

$${V}_{{o}_{2} max} = A + P 1 * S - P 2 * G + P 3 * V * \frac{P 4}{B} - C * \frac{{HR}_{chara} - {HR}_{rest}}{{HR}_{max} - {HR}_{rest}} - \frac{2 ( a - 26 )}{5} ,$$

wherein A is a constant from 40 to 50, P1 is a constant from 7 to 8, S is a gender constant, male is equal to 1, and female is equal to 0; P2 is a constant from 0.1 to 0.2, G is the user's weight; P3 is 4 to 5, V is the characteristic average speed, P4 is a constant from 3 to 4, B is a constant from 1 to 2; C is a constant from 15 to 20, HR_{chara} is the characteristic average heart rate, HRᵣₑₛₜ is the resting heart rate in a quiet state for the user, HRₘₐₓ is the maximum heart rate; and a is the user's age.

Preferably, in the foregoing embodiment of obtaining the maximal oxygen consumption, in order to make the calculated maximal oxygen consumption close to the actual level to the greatest extent, the real-time heart rate and real-time exercise speed can be filtered through the following steps:

S400, determining whether the user's single continuous exercise duration T3 is greater than a preset duration T4, if yes, perform step S401; otherwise, return; in the present embodiment, T4 is equal to 20 minutes.

S401, in a certain movement period, according to time sequence, extracting the real-time heart rate and the real-time exercise speed in each continuous unit time period (I minute) and determining whether all real-time heart rates are within a numerical interval D3 of a preset maximum heart rate, if yes, then the real-time heart rate and real-time exercise speed in the current unit time period are included in the range of data collection; otherwise, the current collection is discarded. In this embodiment, the numerical interval D3 is [70%*HRₘₐₓ, 95%*HRₘₐₓ] preferably.

For the selection of the characteristic time period, preferably, when the user's continuous exercise time T3 is greater than 30 minutes, the characteristic time period is a period between 10 and 30 minutes, and when the user's continuous exercise time T3 is less than or equal to 30 minutes, the characteristic time period is a period between 10 and T3.

In a preferable embodiment, the method for evaluating a physical stamina indicator in running exercise further includes a method for evaluating physical stamina conditions, and the method includes:

defining several different evaluation characteristics that indicate physical stamina condition; For example, defining the physical stamina condition from poor to optimum as poor, weak, fair, good, and optimum;

acquiring user's a second standard pace that is relevant to the standard schedule (such as half marathon) and reflects user's running level in recent M (such as 28) days, and obtaining a second physical stamina level reflecting user's athletic ability through the first model equation, wherein M>N;

calculate a physical stamina difference DV between the second physical stamina level and the first physical stamina level;

establishing a physical stamina condition evaluation table which records the evaluation characteristics corresponding to users with different physical stamina differences DV; and

querying the physical stamina condition evaluation table according to use's physical stamina difference DV, thereby obtaining user's evaluation characteristics representing the physical stamina condition.

Specifically, the obtaining process of the second standard pace can be referred to the process of obtaining the first standard pace. First, based on the 28-day exercise condition data, a third model equation reflecting the relationship between the user's heart rate and pace is established: y₃=k₃*x₃+b₃, wherein k₃ and b₃ are constants, y₃ represents the exercise heart rate of the user's potential ability, and x₁ represents the running pace of the user's potential ability. Since the second model equation is fitted with 7-day exercise state data, it is a regression model of athletic performance, and the third model equation is fitted with 28-day exercise state data, so it is a regression model of exercise performance. The first physical stamina level can be obtained through the first model equation and the second model equation, the second physical stamina level can be obtained through the first model equation and the third model equation, and then the physical stamina difference (DV) between the first physical stamina level and the second physical stamina level can be calculated, and then the user's current physical stamina condition is obtained by querying the physical stamina condition evaluation table, as shown in Table 6 below.

TABLE 6

| Physical stamina | Physical stamina |
|---|---|
| difference DV | condition |
| (4, +∞) | poor |
| (1, 4] | weak |
| (−1, 1] | fair |
| (−4, −1] | good |
| (−6, −4] | optimum |

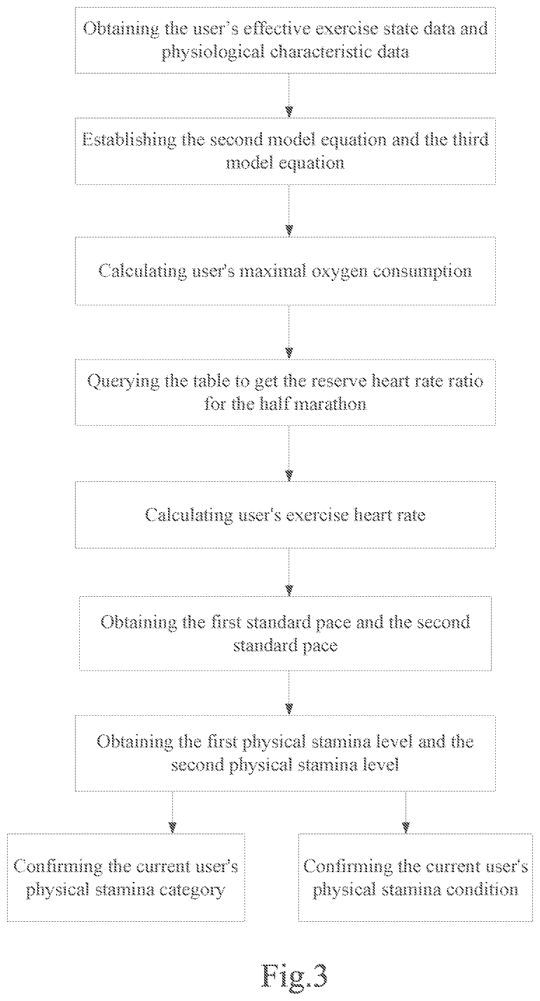

In summary, as shown in FIG. 3, the implementation process of the physical stamina indicator evaluation method in the running exercise of the present invention for the physical stamina level assessment and the physical stamina condition assessment includes:

First, obtaining the user's effective exercise state data and physiological characteristic data, collecting the current user's 7-day and 28-day exercise state data, respectively, establishing the second model equation and the third model equation; then, calculating the user's maximal oxygen consumption according to the user's physiological characteristic data and calculation formulas; then, according to the user's gender characteristics and maximal oxygen consumption, querying the table (Table 4 or Table 5) to get the user's appropriate reserve heart rate ratio for the half marathon; then, according to the calculation formula of the reserve heart rate ratio, obtaining the user's exercise heart rate which is substituted into the second model equation and the third model equation to obtain the first standard pace and the second standard pace; and then obtaining the first physical stamina level and the second physical stamina level according to the first model equation; finally, querying the table (Table 1 or Table 2) according to the first physical stamina level to confirm the current user's physical stamina category, and querying the table (Table 6) according the difference DV between the two levels to confirm the current user's physical stamina condition. In such a way, the evaluation result is highly accurate and can be referenced, and for the user, the intuitive physical stamina level indicator and the physical stamina condition indicator can be obtained easily by only providing the relevant basic data, the operation is convenient.

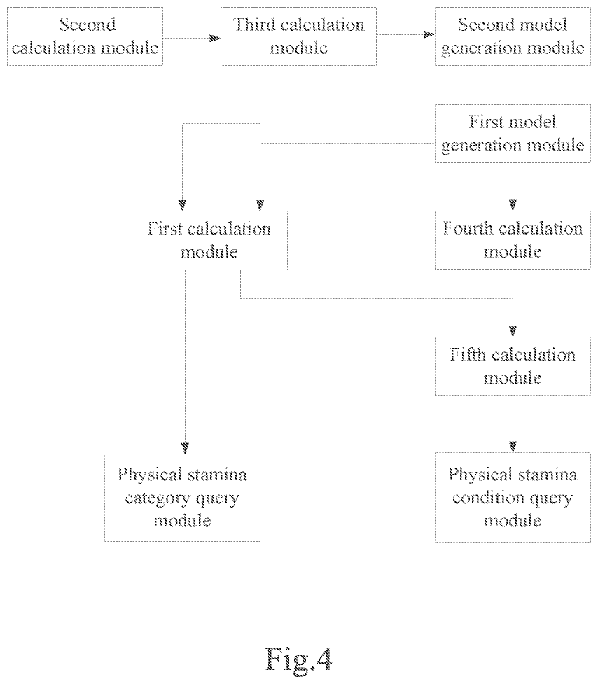

Referring to FIG. 4, a device for evaluating a physical stamina indicator in running exercise according to the present invention includes a physical stamina category evaluation unit and a physical stamina condition evaluation unit. Specifically, the physical stamina category evaluation unit includes a physical stamina level quantification module, a first model generation module, a first calculation module, and a physical stamina category query module. More specifically, the physical stamina level quantification module is configured to quantify a size of physical stamina level by using a closed numerical interval D1 that is defined. The first model generation module is configured to establish a first model equation corresponding to a certain standard schedule, where the first model equation reflects a relationship between a physical stamina level and a pace under the standard schedule. The first calculation module is configured to acquire user's a first standard pace that is relevant to the standard schedule and reflects user's running level in recent N days, and obtain a first physical stamina level reflecting user's exercise performance through the first model equation. The physical stamina category query module is configured to query a physical stamina category query table according to the first physical stamina level, thereby obtaining user's physical stamina level. As a preferable embodiment, the physical stamina category evaluation unit further includes a second model generation module, a second calculation module, and a third calculation module. Specifically, the second model generation module is configured to establish a second model equation reflecting a relationship between user's heart rate and pace, according to a real-time heart rate and a real-time exercise speed of the user during the exercise in recent N days; the second calculation module is configured to calculate user's exercise heart rate for the standard schedule according to user's maximum heart rate, resting heart rate and reserve heart rate ratio; and the third calculation module is configured to calculate user's first standard pace for the standard schedule according to the exercise heart rate and the second model equation.

The physical stamina condition evaluation unit includes a fourth calculation module, a fifth calculation module, and a physical stamina condition query module. Specifically, the fourth calculation module is configured to acquire user's a second standard pace that is relevant to the standard schedule and reflects user's running level in recent M days, and calculate a second physical stamina level that reflects user's athletic ability through the first model equation, wherein M>N. The fifth calculation module is configured to obtain a physical stamina difference DV between the second physical stamina level and the first physical stamina level. The physical stamina condition query module is configured to obtain the current user's evaluation characteristics indicating the physical stamina condition by querying the physical stamina condition evaluation table according to the current physical stamina difference DV. In addition, the device for evaluating the physical stamina indicator in running exercise in this embodiment is also provided with a heart rate sensor and a GPS system to detect the heart rate and moving speed of the user during exercise.

For the working principle of the physical stamina indicator evaluation device in running exercise in the above embodiment, please refer to the above-mentioned method for evaluating physical stamina indicators in running exercise, which will not be repeated here.

Additionally, the present invention discloses an apparatus for evaluating physical stamina indicators in running exercise, including one or more processors; one or more memories; and one or more programs, stored in said one or more memories and configured to be executed by said one or more processors, for executing the method for evaluating physical stamina indicators in running exercise mentioned above.

The present invention also discloses a readable storage medium in which programs are stored. When the programs are executed by the processor, to implement the method for evaluating physical stamina indicators in running exercise as described above.

While the invention has been described in connection with what are presently considered to be the most practical and preferred embodiments, it is to be understood that the invention is not to be limited to the disclosed embodiments, but on the contrary, is intended to cover various modifications and equivalent arrangement included within the spirit and scope of the invention.

1-16. (canceled)

17. A method for evaluating a physical stamina category in running exercise, comprising:
obtaining a first standard pace of a user;
obtaining a first physical stamina level based on the first standard pace through a first model equation, the first model equation reflecting a relationship between physical stamina level and pace;
obtaining physiological characteristics of the user; and
obtaining the user's physical stamina category based on the first physical stamina level and the physiological characteristics by querying a physical stamina category query table, the physical stamina category query table recording stamina levels corresponding to different physiological characteristics and different physical stamina categories.

18. The method as claimed in claim 17, wherein obtaining the first standard pace of the user comprises:
obtaining an exercise heart rate of the user; and
obtaining the first standard pace based on the exercise heart rate through a second model equation, the second model equation reflecting a relationship between the user's heart rate and pace.

19. The method as claimed in claim 18, wherein the exercise heart rate is acquired based on a heart rate reserve ratio, the maximal heart rate and a resting heart rate of the user.

20. The method as claimed in claim 19, wherein the heart rate reserve ratio is acquired based on the physiological characteristics and maximal oxygen consumption of the user by querying a heart rate reserve ratio query table.

21. The method as claimed in claim 20, wherein the maximal oxygen consumption is acquired based on the user's age, gender, weight, characteristic average speed, characteristic average heart rate, maximal heart rate, and resting heart rate.

22. The method as claimed in claim 17, wherein the first model equation is a regression model based on physical stamina level and pace.

23. The method as claimed in claim 17, further comprising:
obtaining a second standard pace and a second physical stamina level;
calculating the difference between the first physical stamina level and the second physical stamina level; and
obtaining a physical stamina condition of the user based on the difference by querying a physical stamina condition evaluation table.

24. The method as claimed in claim 23, wherein the first standard pace is a pace for an N days exercise, the second standard pace is a pace for an M days exercise, and M is greater than N.

25. The method as claimed in claim 17, wherein the physiological characteristics of the user comprises at least one selected from the group of age, gender and weight.

26. An apparatus for a physical stamina category in running exercise, comprising:
one or more processors;
one or more memories; and
one or more programs, stored in said one or more memories and configured to be executed by said one or more processors, for executing:
obtaining a first standard pace of a user;
obtaining a first physical stamina level based on the first standard pace through a first model equation, the first model equation reflecting a relationship between physical stamina level and pace;
obtaining physiological characteristics of the user; and
obtaining the user's physical stamina category based on the first physical stamina level and the physiological characteristics by querying a physical stamina category query table, the physical stamina category query table recording stamina levels corresponding to different physiological characteristics and different physical stamina categories.

27. The apparatus as claimed in claim 26, wherein obtaining the first standard pace of the user comprises:
obtaining an exercise heart rate of the user; and
obtaining the first standard pace based on the exercise heart rate through a second model equation, the second model equation reflecting a relationship between the user's heart rate and pace.

28. The apparatus as claimed in claim 27, wherein the exercise heart rate is acquired based on a heart rate reserve ratio, the maximal heart rate and a resting heart rate of the user.

29. The apparatus as claimed in claim 28, wherein the heart rate reserve ratio is acquired based on the physiological characteristics and maximal oxygen consumption of the user by querying a heart rate reserve ratio query table.

30. The apparatus as claimed in claim 29, wherein the maximal oxygen consumption is acquired based on the user's age, gender, weight, characteristic average speed, characteristic average heart rate, maximal heart rate, and resting heart rate.

31. The apparatus as claimed in claim 26, wherein the first model equation is a regression model based on physical stamina level and pace.

32. The apparatus as claimed in claim 26, wherein the one or more processors are configured to further execute:
obtaining a second standard pace and a second physical stamina level;
calculating the difference between the first physical stamina level and the second physical stamina level; and
obtaining a physical stamina condition of the user based on the difference by querying a physical stamina condition evaluation table.

33. The apparatus as claimed in claim 32, wherein the first standard pace is a pace for an N days exercise, the second standard pace is a pace for an M days exercise, and M is greater than N.

34. The apparatus as claimed in claim 26, wherein the physiological characteristics of the user comprises at least one selected from the group of age, gender and weight.

35. A computer readable storage medium, comprising computer programs configured to be executed by a processor to implement:
obtaining a first standard pace of a user;
obtaining a first physical stamina level based on the first standard pace through a first model equation, the first model equation reflecting a relationship between physical stamina level and pace;
obtaining physiological characteristics of the user; and
obtaining the user's physical stamina category based on the first physical stamina level and the physiological characteristics by querying a physical stamina category query table, the physical stamina category query table recording stamina levels corresponding to different physiological characteristics and different physical stamina categories.
